(19) United States
(12) Patent Application Publication
(10) Pub. No.: US 2002/0091350 A1
(43) Pub. Date: Jul. 11, 2002

(54) Hemodialysis treatment apparatus and method

(76) Inventor: Amir Belson, Cupertino, CA (US)

Correspondence Address: LEARY & ASSOCIATES, 3900 NEWPARK MALL RD., THIRD FLOOR, SUITE 317, NEWARK, CA, 94560, US

(21) Appl. No.: 09/992,526

(22) Filed: Nov. 13, 2001

Related U.S. Application Data
(60) Provisional application No. 60/248,342, filed on Nov. 13, 2000.

(51) Int. Cl. A61M 37/00; C02F 1/44

(52) U.S. Cl. 604/5.04; 210/646

(57) ABSTRACT

An improved apparatus for hemodialysis treatment includes an ultrasonic module with an ultrasonic transducer coupled to a membrane or hollow fiber dialyzer. An ultrasonic waveform generator is switchable between various modes, including a narrowband sine wave, a variable or sweeping frequency sine wave and a broadband square or sawtooth waveform. A low power setting is used to increase the diffusion rate across the semipermeable membranes of the dialyzer and a high power setting is used intermittently to break up thrombus that may form within the dialyzer. A chamber downstream of the dialyzer has an ultrasonic or optical emboli detector for detecting thrombi or emboli exiting the dialyzer and a focused ultrasonic transducer to break up any emboli in the chamber. A screen or filter at the exit of the chamber prevents thrombi and emboli larger than a certain size from entering the patient's circulatory system.

### CROSS-REFERENCE TO OTHER APPLICATION

This application claims the benefit of U.S. Provisional Application No. 60/248,342, filed Nov. 13, 2000.

### FIELD OF THE INVENTION

The present invention relates to an improved apparatus and methods for hemodialysis treatment of patients with renal disease.

### BACKGROUND OF THE INVENTION

Hemodialysis is a lifesaving treatment for many patients with renal disease.

Hemodialysis replaces the function of the kidneys for purifying the blood by removing waste products and excess fluid from the blood in patients whose kidney function has been permanently or temporarily disabled. The patient's blood is pumped through a membrane or hollow fiber dialyzer where it exchanges fluid and dissolved substances with a dialysate solution by diffuision through a semipermeable membrane. Hemodialysis treatments take approximately three to four hours to perform and the treatments are usually repeated three times a week. This treatment regimen is very time consuming and disruptive of the patient's ability to lead a normal life. Improvements to speed up the hemodialysis process would be very beneficial to the patients and would allow more efficient use of medical resources. Further improvements to the hemodialysis process can be realized by a reduction in the necessity for anticoagulation during hemodialysis treatments.

### SUMMARY OF THE INVENTION

In keeping with the foregoing discussion, the present invention takes the form of an improved hemodialysis apparatus and methods for hemodialysis treatment of patients with renal disease. The apparatus is configured for use with a hemodialysis treatment system, which typically includes a membrane or hollow fiber dialyzer where fluid and dissolved substances are exchanged between the patient's blood and a dialysate solution by diffusion across a semipermeable membrane. The apparatus includes an ultrasonic module, which is configured to deliver ultrasonic energy to the dialyzer to improve the efficiency of the hemodialysis treatment system. The ultrasonic module can be a separate unit with means to attach it to the dialyzer or, alternatively, it can be permanently integrated with the dialyzer into a single unit. The ultrasonic module can be constructed as a piece of durable equipment that is reusable with many disposable or reusable dialyzers for a multiplicity of patients, as a single-patient reusable product or as a single-use disposable product.

In a first embodiment, the ultrasonic module includes an ultrasonic transducer and an acoustic coupling, which is configured to efficiently transmit ultrasonic vibrations from the ultrasonic transducer to the body of the dialyzer. The ultrasonic transducer may utilize any known ultrasonic transducer technology, such as piezoelectric transducers, magnetostrictive transducers or silicon ultrasound transducers. The acoustic coupling is split into a first half and a second half with semicylindrical cutouts that are sized to fit around the body of the dialyzer for good acoustic coupling. The first and second halves of the acoustic coupling are hinged together to facilitate insertion of the dialyzer and a closure device is provided to fasten the ultrasonic module around the dialyzer. Other geometries of the ultrasonic transducer and acoustic coupling may be used with noncylindrical dialyzers, such as flat membrane dialyzers.

In an alternate embodiment, the ultrasonic module includes one or more ultrasonic transducers that transmit ultrasonic waves into the chamber of the hollow fiber dialyzer by way of one or more waveguide rods. The waveguide rods may be textured or faceted or have other geometrical features to promote uniform dispersion of the ultrasonic energy within the chamber. Additionally, the waveguide rods may be constructed in the configuration of a tapered ultrasonic amplifying horn to increase the amplitude of the ultrasonic waves produced by the ultrasonic transducers.

In either embodiment, the ultrasonic transducer is connected to the output of an ultrasonic waveform generator, which may operate in one of several possible modes. The ultrasonic waveform generator may produce a simple narrowband sine wave at a desired frequency, or it may produce a variable or sweeping frequency sine wave. The ultrasonic waveform generator may sweep the frequency within a desired range to find a resonance, and lock onto the resonant frequency. Alternatively, the ultrasonic waveform generator may produce a broadband waveform, such as a square wave or a sawtooth wave. The ultrasonic waveform generator may be made switchable between these various modes for different purposes. The ultrasonic waveform generator may operate over a wide range of frequencies, including sonic frequencies and ultrasonic frequencies in the kilohertz and megahertz ranges. The ultrasonic waveform generator preferably includes a variable power output, with a low power setting for continuous use to increase the diffusion rate across the semipermeable membranes of the dialyzer and a high power setting for intermittent application to break up thrombus that may form within the dialyzer.

In use, the ultrasonic waveform generator energizes the ultrasonic transducer to produce ultrasonic waves at a desired frequency and amplitude and with a desired waveform to increase the diffusion rate across the semipermeable membrane of the dialyzer. The increased diffusion rate significantly reduces the amount of time required for hemodialysis treatments. Intermittently, the power output of the ultrasonic waveform generator may be increased to a higher level to break up any thrombus that may form within the dialyzer and to remove any platelets or fibrin that may have deposited on the surfaces of the semipermeable membrane. The frequency and the waveform, as well as the amplitude of the ultrasonic waves may also be changed. This will keep the dialyzer working at maximum efficiency for a longer period of time. This feature also provides an advantage by reducing or eliminating the necessity for anticoagulation during hemodialysis treatments.

Optionally, the invention may also include a thrombus detection and thrombolysis module. An emboli detector, which may be an ultrasonic or optical detector, detects thrombi or other emboli exiting the dialyzer. When an embolus is detected, a control module energizes an ultrasonic transducer that is focused on a chamber below the dialyzer to break up the embolus. Thrombi and emboli larger than a certain size are prevented from entering the patient's circulatory system by a screen or filter at the exit of the chamber.

### BRIEF DESCRIPTION OF THE DRAWINGS

FIG. 1 is a schematic diagram showing the improved hemodialysis treatment apparatus of the present invention.

FIG. 2 is a side view showing the ultrasonic module of the improved hemodialysis treatment apparatus applied to a hollow fiber dialyzer.

FIG. 3 is a cross section of the ultrasonic module and the hollow fiber dialyzer of FIG. 2.

FIG. 4 shows a second embodiment of the improved hemodialysis treatment apparatus of the present invention.

FIG. 5 is a cross section of the ultrasonic module and the hollow fiber dialyzer of FIG. 4.

### DETAILED DESCRIPTION OF THE INVENTION

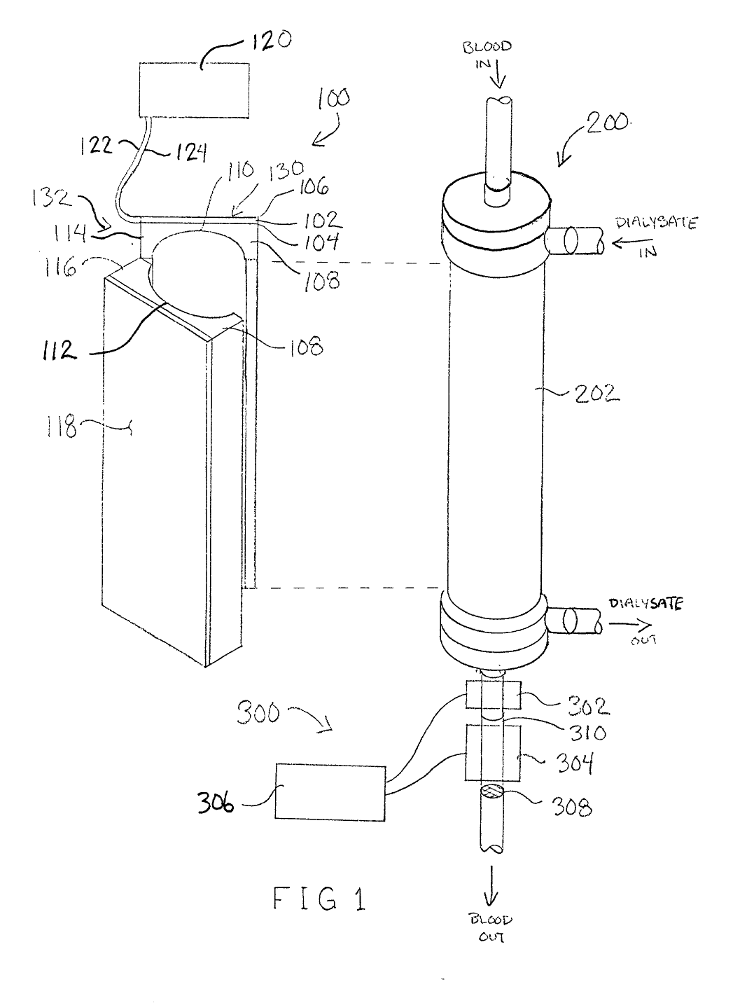

The improved hemodialysis treatment apparatus of the present invention is shown schematically in FIG. 1. The apparatus of the present invention is intended for use with a standard hemodialysis treatment system. The construction and operation of such systems are well known in the art and thus need not be described in detail here. The hemodialysis treatment system will typically include a membrane or hollow fiber dialyzer 200 where fluid and dissolved substances are exchanged between the patient's blood and a dialysate solution by diffuision across a semipermeable membrane. A cross section of a typical hollow fiber dialyzer 200 can be seen in FIG. 3. A semipermeable membrane in the form of a multiplicity of hollow fibers 204 passes through the cylindrical body 202 of the dialyzer 200. Blood flows through the hollow fibers 204 and the dialysate solution flows within a chamber 206 surrounding the hollow fibers 204. Diffusion takes place between the patient's blood and the dialysate solution across the walls of the hollow fibers 204.

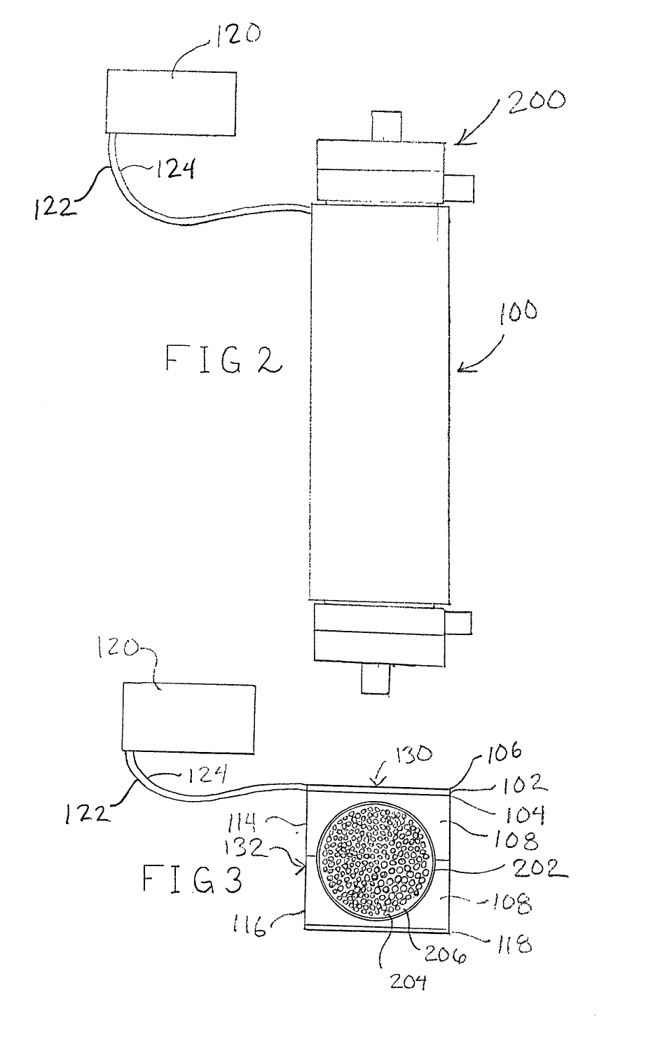

The invention includes an ultrasonic module 100, which is configured to attach to the dialyzer 200 and to improve the efficiency of the hemodialysis treatment system. The ultrasonic module 100 and the dialyzer 200 are shown assembled together in a side view in FIG. 2 and in a cross section in FIG. 3. In one preferred embodiment of the invention, the ultrasonic module 100 can be constructed as a piece of durable equipment that is reusable with many disposable or reusable dialyzers 200 for a multiplicity of patients. In this case, since the ultrasonic module 100 does not directly contact the patient's blood or the dialysate solution, the ultrasonic module 100 would not need to be sterilized between uses. In an alternate preferred embodiment, the ultrasonic module 100 can be permanently integrated with the dialyzer 200 into a single unit. The combined ultrasonic module 100 and dialyzer 200 unit can be constructed as a disposable product or as a single-patient reusable product. In this case, the combined ultrasonic module 100 and dialyzer 200 unit would be constructed so that it can be sterilized before use.

The ultrasonic module 100 includes an ultrasonic transducer 130 and an acoustic coupling 132. The ultrasonic transducer 130 is preferably constructed as of a layer of piezoelectric material 102, which is coated on a first side with a first conductive electrode 104 and on a second side with a second conductive electrode 106. An insulating layer may be coated over the electrodes 104, 106. The piezoelectric material 102 used in the ultrasonic transducer 130 may be a polymeric piezoelectric material, such as polyvinylidene difluoride (PVDF), or a ceramic piezoelectric material, such as lead zirconium titanate (PZT), or other known piezoelectric materials. If desired, the ultrasonic transducer 130 may be constructed with multiple layers of piezoelectric material 102 to increase the amplitude and/or power of the ultrasonic waves produced. Alternatively, the ultrasonic transducer 130 may utilize other known ultrasonic transducer technologies, such as magnetostrictive transducers or silicon ultrasound transducers. Suitable silicon ultrasound transducers, which are produced on silicon wafers using MEMS (Micro Electro Mechanical Systems) technology, are available from Sensant Corporation, 14470 Doolittle Drive, San Leandro, Calif., USA 94577 and are described in U.S. Pat. No. 6,246,158, which is hereby incorporated by reference.

The acoustic coupling 132 is configured to efficiently transmit ultrasonic vibrations from the ultrasonic transducer 130 to the body 202 of the dialyzer 200. For convenience, the acoustic coupling 132 is split into a first half 114 and a second half 116 that hinge apart or separate to facilitate insertion of the body 202 of the dialyzer 200 into the ultrasonic module 100, as shown in FIG. 1. The first and second halves 114, 116 of the acoustic coupling 132 are made primarily of an acoustic coupling material 108 having an acoustic impedance that is matched approximately to the acoustic impedance of the blood and the dialysate solution. Various materials, such as polyurethane, low density polyethylene and gel materials, are suitable for use as an acoustic coupling material 108. The first and second halves 114, 116 of the acoustic coupling 132 have semicylindrical cutouts 110, 112, which are sized to fit tightly around the cylindrical body 202 of the dialyzer 200 for good acoustic coupling when the ultrasonic module 100 is in a closed position, as shown in FIGS. 2 and 3. A latch, clamp or other closure device may be provided to hold the ultrasonic module 100 in the closed position.

Other geometries of the ultrasonic transducer 130 and acoustic coupling 132 are possible, for example for use with noncylindrical dialyzers, such as flat membrane dialyzers. Alternatively, the ultrasonic transducer 130 may be coupled directly to the body 202 or to the chamber 206 of the dialyzer 200 without an additional acoustic coupling 132. For example, this can be accomplished by utilizing an ultrasonic transducer 130 having an acoustic impedance that is matched approximately to the acoustic impedance of the blood and the dialysate solution. Additionally, the ultrasonic transducer 130 may be configured as an array of transducers in any desired geometry. Such a transducer array may be coupled directly to the body 202 or to the chamber 206 of the dialyzer 200 or indirectly through one or more acoustic couplings.

The ultrasonic transducer 130 is attached to the first half 114 of the acoustic coupling 132. Preferably, the second half 116 of the acoustic coupling 132 has an acoustically reflective surface 118 positioned opposite to and parallel with the ultrasonic transducer 130. The acoustically reflective surface 118 may be backed with a high acoustic impedance material, such as a metal, which will produce a positive reflection of the ultrasonic waves, or a low acoustic impedance material, such as air, which will produce a negative reflection of the ultrasonic waves. The acoustically reflective surface 118 allows the acoustic coupling 132 to be designed as a resonant structure, which will increase the efficiency of the ultrasonic transducer 130. In a preferred configuration, the ultrasonic transducer 130 and the acoustic coupling 132 extend substantially the full length of the body 202 of the dialyzer 200. If desired, the ultrasonic transducer 130 and the acoustic coupling 132 may be enclosed in a protective and esthetic housing.

The first electrode 104 and the second electrode 106 of the ultrasonic transducer 130 are connected to the output of an ultrasonic waveform generator 120 by a first electrical lead 122 and a second electrical lead 124, respectively. Preferably, the first electrical lead 122 and the second electrical lead 124 are configured as a coaxial cable. The ultrasonic waveform generator 120 may operate in one of several possible modes. The ultrasonic waveform generator 120 may produce a simple narrowband sine wave at a desired frequency, or it may produce a variable or sweeping frequency sine wave. The ultrasonic waveform generator 120 may sweep the frequency within a desired range to find a resonance, indicated by a local minimum in the electrical impedance, and lock onto the resonant frequency. Alternatively, the ultrasonic waveform generator 120 may produce a broadband waveform, such as a square wave or a sawtooth wave. The ultrasonic waveform generator 120 may be made switchable between these various modes for different purposes. The ultrasonic waveform generator 120 may operate over a wide range of frequencies, including sonic frequencies and ultrasonic frequencies in the kilohertz and megahertz ranges. Ultrasonic frequencies in the range of 20 to 40 kilohertz are thought to be particularly effective for use in the present invention. The ultrasonic waveform generator 120 will preferably include a variable power output, with at least a low power setting for continuous use to increase the diffusion rate across the semipermeable membranes of the dialyzer 200 and a high power setting for intermittent application to break up thrombus that may form within the dialyzer 200.

In use, the ultrasonic waveform generator 120 energizes the ultrasonic transducer 130 to produce ultrasonic waves at a desired frequency and amplitude and with a desired waveform to increase the diffusion rate across the semipermeable hollow fiber membranes 204 of the dialyzer 200. The increased diffusion rate significantly reduces the amount of time required for hemodialysis treatments. The ultrasonic waves are transmitted from the ultrasonic transducer 130 into the body 202 of the dialyzer 200 by the acoustic coupling 132, preferably producing a uniform acoustic field within the chamber 206 of the dialyzer 200. Intermittently, the power output of the ultrasonic waveform generator 120 may be increased to a higher level to break up any thrombus that may form within the dialyzer 200 and to remove any platelets or fibrin that may have deposited on the surfaces of the hollow fiber membranes 204. The frequency and the waveform, as well as the amplitude of the ultrasonic waves may also be changed. This will keep the dialyzer 200 working at maximum efficiency for a longer period of time. This feature also provides an advantage by reducing or eliminating the necessity for anticoagulation during hemodialysis treatments.

Optionally, the invention may also include a thrombus detection and thrombolysis module 300, as shown in FIG. 1. An emboli detector 302, which may be an ultrasonic or optical detector, detects thrombi or other emboli exiting the dialyzer 200. When an embolus is detected, a control module 306 energizes an ultrasonic transducer 304 that is focused on a chamber 310 below the dialyzer 200 to break up the embolus. Thrombi and emboli larger than a certain size are prevented from entering the patient's circulatory system by a screen or filter 308 at the exit of the chamber 310.

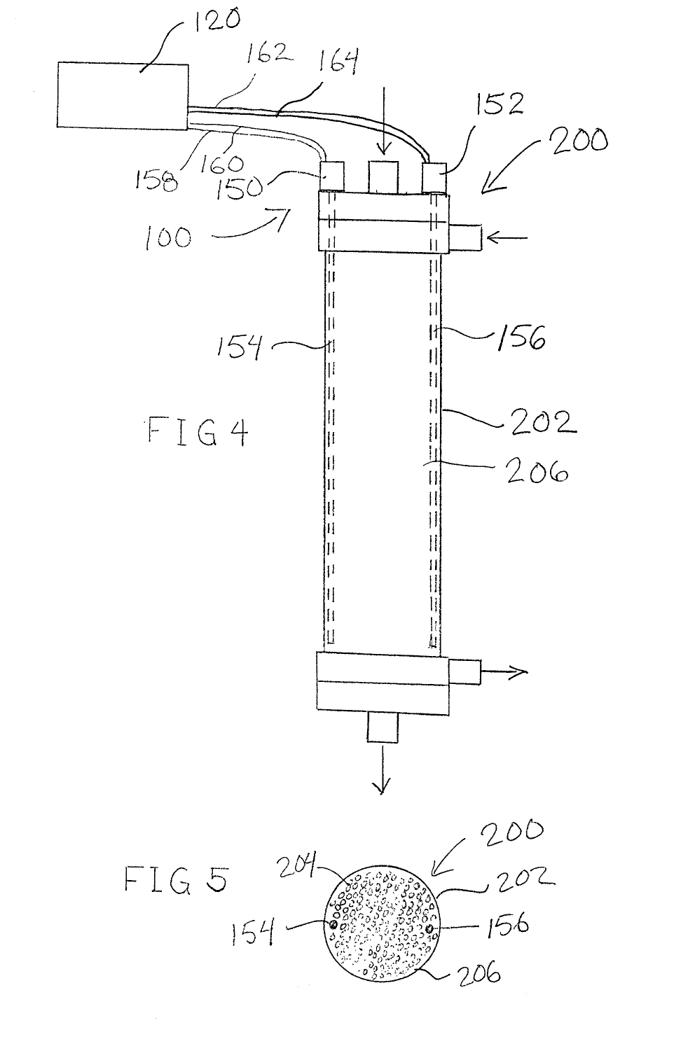

FIG. 4 shows a second embodiment of the improved hemodialysis treatment apparatus, which includes an ultrasonic module 100 and a hollow fiber dialyzer 200. FIG. 5 is a cross section of the ultrasonic module 100 and the hollow fiber dialyzer 200 of FIG. 4. In this embodiment, the ultrasonic module 100 takes the form of one or more ultrasonic transducers 150, 152 that transmit ultrasonic waves into the chamber 206 of the hollow fiber dialyzer 200 by way of one or more waveguide rods 154, 156. The ultrasonic transducers 150, 152 may utilize piezoelectric transducers, magnetostrictive transducers, silicon ultrasound transducers or other known ultrasonic transducer technologies. The waveguide rods 154, 156 are preferably constructed of a metal or other material that will efficiently conduct the ultrasonic energy into the chamber 206 of the hollow fiber dialyzer 200 and transfer the ultrasonic waves to the dialysate solution. Suitable materials for the waveguide rods 154, 156 include, but are not limited to, stainless steel, titanium, titanium alloys and cobalt alloys. The waveguide rods 154, 156 may be textured or faceted or have other geometrical features to promote uniform dispersion of the ultrasonic energy within the chamber 206. The waveguide rods 154, 156 may also be constructed in the configuration of a tapered ultrasonic amplifying horn to increase the amplitude of the ultrasonic waves produced by the ultrasonic transducers 150, 152.

By way of example, FIG. 4 shows the apparatus with two such ultrasonic transducers 150, 152 connected to two waveguide rods 154, 156. Alternatively, the apparatus may be constructed with a single ultrasonic transducer connected to one or more waveguide rods or with multiple ultrasonic transducers and waveguide rods. Preferably, the waveguide rods 154, 156 are arranged to produce a relatively uniform acoustic field within the chamber 206.

In one preferred embodiment of the invention, the ultrasonic module 100 can be permanently integrated with the dialyzer 200 into a single unit. The combined ultrasonic module 100 and dialyzer 200 unit can be constructed as a disposable product or as a single-patient reusable product. In this case, the combined ultrasonic module 100 and dialyzer 200 unit would be constructed so that it can be sterilized before use. In an alternate preferred embodiment, the ultrasonic module 100 can be constructed as a piece of durable equipment that is reusable with many disposable or reusable dialyzers 200 for a multiplicity of patients. In this case, since the waveguide rods 154, 156 of the ultrasonic module 100 do contact the dialysate solution, the ultrasonic module 100 would be constructed so that it could be sterilized between uses.

The ultrasonic transducers 150, 152 are connected to the output of the ultrasonic waveform generator 120 by electrical leads 158 & 160 and 162 & 164, respectively. Preferably, the electrical leads are configured as coaxial cables. Preferably, the ultrasonic transducers 150, 152 operate at the same frequency and in phase with one another to produce a relatively uniform and constant acoustic field. Alternatively, the ultrasonic transducers 150, 152 may be operated at different frequencies and/or out of phase with one another to produce different acoustic effects within the chamber 206. As described above, the ultrasonic waveform generator 120 may operate in one of several possible modes, including narrowband and broadband modes, and with low and high power settings.

In use, the ultrasonic waveform generator 120 energizes the ultrasonic transducers 150, 152 to produce ultrasonic waves at a desired frequency and amplitude and with a desired waveform to increase the diffusion rate across the semipermeable hollow fiber membranes 204 of the dialyzer 200. The increased diffusion rate significantly reduces the amount of time required for hemodialysis treatments. The ultrasonic waves are transmitted from the ultrasonic transducers 150, 152 into the chamber 206 of the dialyzer 200 by the waveguide rods 154, 156, which serve as an acoustic coupling to the dialysate solution. Intermittently, the power output of the ultrasonic waveform generator 120 may be increased to a higher level to break up any thrombus that may form within the dialyzer 200 and to remove any platelets or fibrin that may have deposited on the surfaces of the hollow fiber membranes 204. The frequency and the waveform, as well as the amplitude of the ultrasonic waves may also be changed. This will keep the dialyzer 200 working at maximum efficiency for a longer period of time. This feature also provides an advantage by reducing or eliminating the necessity for anticoagulation during hemodialysis treatments. Optionally, the invention may also be used with the thrombus detection and thrombolysis module 300 described above in connection with FIG. 1.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

1. Apparatus for hemodialysis treatment, comprising:
a dialyzer having a flow path for a patient's blood and a flow path for a dialysate solution and a semipermeable membrane configured to separate the blood flow path from the dialysate flow path; and
an ultrasonic transducer configured to deliver ultrasonic energy into the dialyzer.

2. The apparatus of claim 1, wherein:
the semipermeable membrane of the dialyzer is configured as a hollow fiber membrane.

3. The apparatus of claim 1, further comprising:
an acoustic coupling for coupling ultrasonic energy from the ultrasonic transducer into the dialyzer.

4. The apparatus of claim 3, wherein:
the dialyzer is configured with a cylindrical body; and
the acoustic coupling is configured with two halves, each of the two halves having an approximately semicylindrical cutout configured to fit around the cylindrical body of the dialyzer.

5. The apparatus of claim 1, further comprising:
at least one waveguide rod for coupling ultrasonic energy from the ultrasonic transducer into the dialyzer.

6. The apparatus of claim 1, further comprising:
an ultrasonic waveform generator connected to the ultrasonic transducer.

7. The apparatus of claim 6, wherein:
the ultrasonic waveform generator is switchable between modes, including a narrowband sine wave, a variable or sweeping frequency sine wave and a broadband square or sawtooth waveform.

8. The apparatus of claim 6, wherein:
the ultrasonic waveform generator is configured to vary the frequency within a desired range to find a resonant frequency and to lock onto the resonant frequency.

9. The apparatus of claim 6, wherein:
the ultrasonic waveform generator has a low power setting effective to increase the diffusion rate across the semipermeable membrane of the dialyzer and a high power setting effective to break up thrombus that forms within the dialyzer.

10. The apparatus of claim 1, further comprising:
a chamber downstream of the dialyzer;
an emboli detector for detecting thrombi and emboli entering the chamber from the dialyzer; and
an ultrasonic transducer configured to break up thrombi and emboli in the chamber.

11. The apparatus of claim 10, further comprising:
a filter configured to prevent thrombi and emboli larger than a predetermined size from entering the patient's circulatory system from the chamber.

12. Apparatus for hemodialysis treatment, comprising:
a dialyzer having a flow path for a patient's blood and a flow path for a dialysate solution and a semipermeable membrane configured to separate the blood flow path from the dialysate flow path;
a chamber downstream of the dialyzer;
an emboli detector for detecting thrombi and emboli entering the chamber from the dialyzer; and
an ultrasonic transducer configured to break up thrombi and emboli in the chamber.

13. The apparatus of claim 12, further comprising:
a filter configured to prevent thrombi and emboli larger than a predetermined size from entering the patient's circulatory system from the chamber.

14. A method of hemodialysis treatment, comprising:
connecting a patient's circulatory system to a dialyzer having a flow path for the patient's blood and a flow path for a dialysate solution and a semipermeable membrane configured to separate the blood flow path from the dialysate flow path; and
delivering ultrasonic energy into the dialyzer.

15. The method of claim 14, wherein:
ultrasonic energy is delivered into the dialyzer at a power level effective to increase the diffusion rate across the semipermeable membrane of the dialyzer.

16. The method of claim 14, wherein:
ultrasonic energy is delivered into the dialyzer at a power level effective to break up thrombus that forms within the dialyzer.

17. The method of claim 14, wherein:
ultrasonic energy is delivered into the dialyzer at a power level effective to increase the diffusion rate across the semipermeable membrane of the dialyzer; and
ultrasonic energy is intermittently delivered into the dialyzer at a power level effective to break up thrombus that forms within the dialyzer.

18. The method of claim 14, further comprising:
detecting thrombi and emboli entering a chamber downstream of the dialyzer; and
energizing an ultrasonic transducer to break up thrombi and emboli in the chamber.
